(19) United States
(12) Patent Application Publication
(10) Pub. No.: US 2004/0223930 A1
(43) Pub. Date: Nov. 11, 2004

(54) [ LIP SEAL METHODS FOR PREVENTION OF SNORE AND MOUTH DRYING ]

(76) Inventor: Sie Gearl Cho, Luverne, AL (US)

Correspondence Address: SIE GEARL CHO, 423 WEST 3RD STREET, LUVERNE, AL, 36049, US

(21) Appl. No.: 10/707,370

(22) Filed: Dec. 9, 2003

Related U.S. Application Data
(60) Provisional application No. 60/437,466, filed on Dec. 31, 2002.

(51) Int. Cl. A61K 7/025

(52) U.S. Cl. 424/64

(57) ABSTRACT

This invention of the methods of lip sealing for prevention of snore and for prevention of mouth drying while sleeping. This lip sealing methods are the very effective and the most economical methods in preventing snore and preventing mouth drying while sleeping.

### CROSS REFERENCE TO RELATED APPLICATIONS

This application is the continuing non-provisional application for the provisional application of 60/437,466 dated Dec. 31, 2002.

### BACKGROUND OF INVENTION

It has been a problem for majority of adult human to have snoring when they sleep. This snore problem comes from the opened mouth in unconscious status while sleeping. This opened mouth not only makes snoring but, it also creates a lot of health problems like drying mucous membrane inside mouth, making thick phlegm and sanitary problem like shedding saliva on the pillows. There have been many inventions to treat this snore problem and most of them are to hold the tongue upward so that it could provide more air flow to the throat. Some other inventions are to implant certain devices in the soft palette on the nose to enlarge air passage and increase air amount flowing through nostrils. All these methods were costly and not easy to have the devices made. These devices also have emotional objection to wear and the devices are not easy to use. What the worse thing is that those devices are not working in full satisfaction. There is one method of sealing the mouth with fabric tape, but this device is not well accepted by the users either, because of the emotional objection to wear the tape on their mouth. I have tried to make a device to seal the mouth while in sleep and had many experiments. I found the best way to seal the mouth is to seal the lips with the following invention methods.

### SUMMARY OF INVENTION

As the snoring and mouth drying comes from the opened mouth while sleeping, the most effective method to prevent snore and mouth drying in sleep is to seal the mouth while sleeping.

The best way to seal the mouth while sleeping is; (1) to seal the lips with fast setting non-toxic gel type adhesive material that will be flexible after setting and can be peeled off after the use. This non-toxic gel type material can be packaged either in bottles or in tubes for use. (2) The other method to seal the lips is to apply adhesive material, rubbing the lipstick like adhesive material on the lips. This adhesive material should be non-toxic and should be strong enough to hold the lips closed during the sleep and should be washed off with water after the use.

In either case of above two methods, either in gel type or in lipstick like material, the adhesive material should be applied leaving about 1/2 inch in the center part of the lips to enable cough and sneeze to come out. These adhesive materials should be non-toxic and strong enough to hold the lips closed during the sleep but those should be peeled off or washed off after the use.

These two methods are the most effective and easy to use. And these methods are the most economical in expense and the simplest methods to use. These methods also give comfortable and good appearance for the users.

### BRIEF DESCRIPTION OF DRAWINGS

FIG. 1 is the bottle example for packaging gel type lip sealing material. The cap of this bottle must have a small brush attached on to it to provide easy application of the gel on the lips.

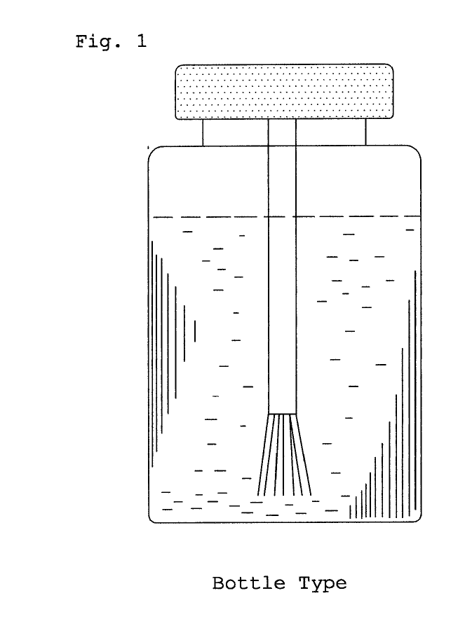

FIG. 2 is the tube example for packaging gel type lip sealing material. To prevent early setting of the gel type material before using, this tube package can be a choice of packaging the gel type material.

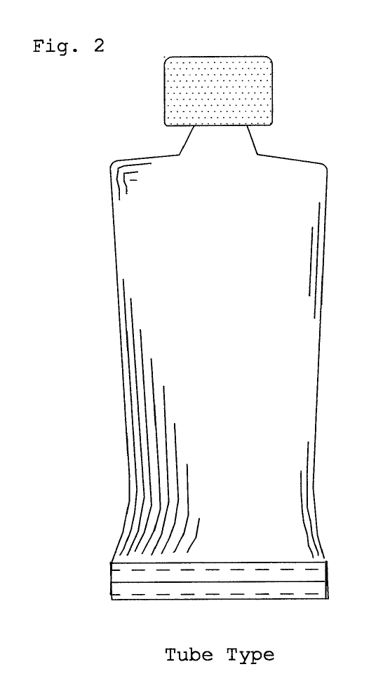

FIG. 3 is the lipstick like adhesive material packaging example. This package type is the most convenient type for applying the adhesive material on the lips. And this package is the most economical, also.

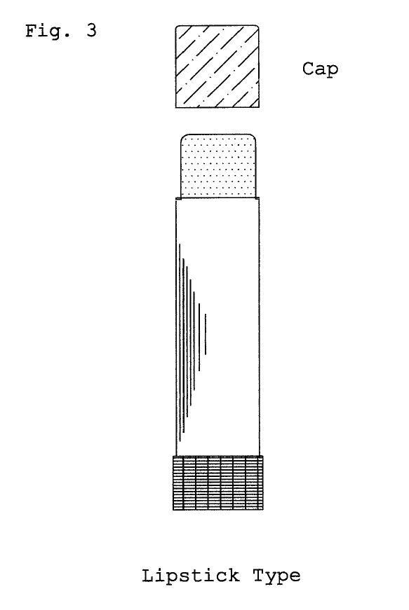

### DETAILED DESCRIPTION

These materials seal the mouth while sleeping so that it prevents snoring and shedding saliva on the pillow, making no dry mucous membrane inside the mouth. Therefore, it prevents weakening the gum and the teeth also, and prevents generation of thick phlegm, too.

[Lip Seal Materials]

| Description | Specification | Package Form |
|---|---|---|
| Gel Type | Non-toxic, Water-base, flexible, | Tube, Bottle |
| | temporary, peel off | |
| Soap Bar Type | non-toxic, temporary, re- | Lipstick |
| | sealable, washable | |

The formulation of gel type lip seal material example is;

A. about 20 percent isopropyl alcohol

B. about 30 percent de-ionized water

C. about 25 percent to 30 percent hydrolyzed polyvinyl alcohol

D. about 5 percent to 10 percent water soluble plasticizer

E. about 5 percent to 10 percent xanthein gum

F. about 3 percent to 5 percent ascorbic acid

G. about 0.2 percent to 0.3 percent orange flavor

The formulation of soap bar type lipstick material example is;

A. about 20 percent beeswax

B. about 30 percent aloe extract

C. about 25 percent to 30 percent petroleum jelly

D. about 5 percent to 10 percent mineral oil

E. about 5 percent to 10 percent xanthein gum

F. about 3 percent to 5 percent candela wax

G. about 2 percent to 3 percent castor oil

H. about 3 percent to 5 percent paraffin

I. about 3 percent to 5 percent ascorbic acid

J. about 0.2 percent to 0.3 percent orange flavor

What I claim as my invention is:

1. The method of using gel type non-toxic water base adhesive materials on human lips for sealing the mouth while sleeping to prevent snoring and mouth drying.

2. The method of packaging the gel type materials of above claim 1 in the bottles.

3. The method of packaging the gel type materials of above claim 1 in the tubes.

4. The method of using non-toxic water base adhesive materials on human lips for sealing the mouth which can be washed off or peeled off after the use.

5. The method of using non-toxic washable soap bar type lipstick adhesive materials on human lips for prevention of snore and to prevent mouth drying while sleeping.

6. The method of using the lipstick type lip sealing materials of above claim 5 packaged in lipstick type type package.

7. The method of using the lipstick type sealing adhesive materials of above claim 5 which will be washed off or peeled off after the use.
